(19) United States
(12) Patent Application Publication
(10) Pub. No.: US 2006/0246524 A1
(43) Pub. Date: Nov. 2, 2006

(54) Nanoparticle conjugates

(76) Inventors: Christina Bauer, Livermore, CA (US); Christopher Bieniarz, Tucson, AZ (US); Anthony L. Hartman, Tucson, AZ (US)

Correspondence Address: VENTANA MEDICAL SYSTEMS, INC.;ATTENTION: LEGAL DEPARTMENT, 1910 INNOVATION PARK DRIVE, TUCSON, AZ, 85755, US

(21) Appl. No.: 11/413,778

(22) Filed: Apr. 28, 2006

Related U.S. Application Data
(60) Provisional application No. 60/675,759, filed on Apr. 28, 2005.
(60) Provisional application No. 60/693,647, filed on Jun. 24, 2005.

(51) Int. Cl. G01N 33/53; C07K 16/46; C07D 403/02

(52) U.S. Cl. 435/7.92; 530/391.1; 548/518; 977/900

(57) ABSTRACT

Conjugate compositions are disclosed that include a specific-binding moiety covalently coupled to a nanoparticle through a heterobifunctional polyalkyleneglycol linker. In one embodiment, a conjugates is provided that includes a specific-binding moiety and a fluorescent nanoparticle coupled by a heterobifunctional PEG linker. Fluorescent conjugates according to the disclosure can provide exceptionally intense and stable signals for immunohistochemical and in situ hybridization assays on tissue sections and cytology samples, and enable multiplexing of such assays.

### RELATED APPLICATION DATA

This application claims the benefit of U.S. Provisional Patent Application No. 60/675,759, filed Apr. 28, 2005, and the benefit of U.S. Provisional Patent Application No. 60/693,647, filed Jun. 24, 2005, both of which applications are incorporated by reference herein.

### BACKGROUND OF THE INVENTION

1. Field

The present invention relates to reagents and methods for detecting a particular molecule in a biological sample. More particularly, the present invention relates to covalent conjugates of specific-binding moieties and nanoparticles as well as methods for using such conjugates to detect particular molecules in biological samples such as tissue sections.

2. Background

Conjugates of specific-binding moieties and signal-generating moieties can be used in assays for detecting specific target molecules in biological samples. The specific-binding portion of such conjugates binds tightly to a target in the sample and the signal-generating portion is utilized to provide a detectable signal that indicates the presence/and or location of the target.

One type of detectable conjugate is a covalent conjugate of an antibody and a fluorophore. Directing photons toward the conjugate that are of a wavelength absorbed by the fluorophore stimulates fluorescence that can be detected and used to qualitate, quantitate and/or locate the antibody. A majority of the fluorescent moieties used as fluorophores are organic molecules having conjugated pi-electron systems. While such organic fluorophores can provide intense fluorescence signals, they exhibit a number of properties that limit their effectiveness, especially in multiplex assays and when archival test results are needed.

Organic fluorophores can be photo-bleached by prolonged illumination with an excitation source, which limits the time period during which maximal and/or detectable signals can be retrieved from a sample. Prolonged illumination and/or prolonged exposure to oxygen can permanently convert organic fluorophores into non-fluorescent molecules. Thus, fluorescence detection has not been routinely used when an archival sample is needed.

Multiplex assays using organic fluorophores are difficult because such fluorophores typically emit photons that are of only slightly greater wavelength (lower energy) than the photons that are aborbed by the fluorophore (i.e., they have a small Stokes shift). Thus, selection of a set of fluorophores that emit light of various wavelengths across a portion of the electromagnetic spectrum (such as the visible portion) requires selection of fluorophores that absorb across the portion. In this situation, the photons emitted by one fluorophore can be absorbed by another fluorophore in the set, thereby reducing the assay's accuracy and sensitivity.

While some organometallic fluorophores (for example, lanthanide complexes) appear to be more photostable than organic fluorophores, sets of them also suffer from overlap of absorption and fluorescence across a region of the spectrum. A further shared shortcoming of organic and organometallic fluorophores is that their fluorescence spectra tend to be broad (i.e. the fluorescent photons span a range of wavelengths), making it more likely that two or more fluorophores in a multiplexed assay will emit photons of the same wavelength. Again, this limits the assay's accuracy. Even in semi-quantitative and qualitative assays these limitations of organic and organometallic fluorophores can skew results.

Fluorescent nanoparticles, for example, fluorescent Cd/Se nanoparticles, are a new class of fluorophores showing great promise for multiplex assays. As part of a broader effort to engineer nanomaterials that exhibit particular properties, fluorescent nanoparticles have been developed to emit intense fluorescence in very narrow ranges of wavelengths. Fluorescent nanoparticles also are highly photostable and can be tuned to fluoresce at particular wavelengths. By virtue of the absorption and fluorescence properties of such nanoparticles, sets of fluorescent nanoparticles that span a wide total range of wavelengths can be simultaneously excited with photons of a single wavelength or within a particular wavelength range (such as in the case of broadband excitation with a UV source) and yet very few or none of the fluorescent photons emitted by any of the particles are absorbed by other nanoparticles that emit fluorescence at longer wavelengths. As a result, fluorescent nanoparticles overcome the limitations of organic and organometallic fluorophores with regard to signal stability and the potential to multiplex an assay.

Some problems arise, however, when nanoparticles generally, and fluorescent nanoparticles specifically, are conjugated to a specific-binding moiety such as an antibody. Surface interactions tend to alter nanoparticle properties. Therefore, conjugation of a nanoparticle to a specific-binding moiety can alter nanoparticle properties and stability, and in the case of fluorescent nanoparticles, their fluorescence properties (such as fluorescence wavelength and intensity). Likewise, interactions between a nanoparticle and a specific-binding moiety can reduce the binding moiety's specificity. Thus, although fluorescent nanoparticles offer a number of properties that make them an attractive alternative to traditional fluorophores, their potential as useful signal-generating moieties in conjugates has not yet been fully realized.

In applications for in situ assays such as immunohistochemical (IHC) assays and in situ hydribization (ISH) assays of tissue and cytological samples, especially multiplexed assays of such samples, it is highly desirable to develop conjugates of fluorescent nanoparticles that retain to a large extent the specificity of the specific-binding moiety and the fluorescence properties of the fluorescent nanoparticles. Retention of these characteristics in a conjugate is even more important when an assay is directed toward detecting low abundance proteins and low copy number nucleic acid sequences.

The unique tunability of the narrow (FWHM<40 nm) quantum dot fluorescence, which can be excited by one excitation source, is extremely attractive for imaging. To this end, quantum dots as analytes have been used in many different architectures. Both electrostatic and covalent bonding have been used for encapsulation of individual quantum dots to prevent aggregation and provide terminal reactive groups. Examples include the use of an amine or carboxyl group for bioconjugation with cross-linking molecules, either through electrostatic interactions or covalent linkage. See for example Chan and Nie “Quantum Dot Bioconjugates for Ultrasensitive Nonisotopic Detection” Science, Vol. 281, 1998, p. 2016-2018 and M. P. Bruchez, et. al. “Method of Detecting an Analyte in a Sample Using Semiconductor Nanocrystals as Detectable Label” U.S. Pat. No. 6,630,307. However, most current methods of making conjugates result in quantum dots where quantum yields are lowered and both stability and archivability is not possible. Therefore, a need exists for conjugates that better retain both the specificity of a specific binding moiety and the desirable photophysical characteristics of the nanoparticle (such as photostability and quantum yield).

### SUMMARY OF THE INVENTION

Conjugates of specific-binding moieties and nanoparticles are disclosed, as are methods for making and using the conjugates. The disclosed conjugates exhibit superior performance for detection of molecules of interest in biological samples, especially for detection of such molecules of interest in tissue sections and cytology samples. In particular, disclosed conjugates of specific binding moieties and fluorescent nanoparticles retain the specificity of the specific binding moieties and the desirable fluorescence characteristics of the nanoparticles, thereby enabling sensitive multiplexed assays of antigens and nucleic acids.

In one aspect, a conjugate is disclosed that includes a specific-binding moiety covalently linked to a nanoparticle through a heterobifunctional polyalkyleneglycol linker such as a heterobifunctional polyethyleneglycol (PEG) linker. In one embodiment, a disclosed conjugate includes an antibody and a nanoparticle covalently linked by a heterobifunctional PEG linker. In another embodiment, a disclosed conjugate includes an avidin and a nanoparticle covalently linked by a heterobifunctional PEG linker. In more particular embodiments, disclosed conjugates include an antibody or an avidin covalently linked to a quantum dot by a heterobifunctional PEG linker.

The PEG linker of disclosed conjugates can include a combination of two different reactive groups selected from a carbonyl-reactive group, an amine-reactive group, a thiol-reactive group and a photo-reactive group. In particular embodiments, the PEG linker includes a combination of a thiol reactive group and an amine-reactive group or a combination of a carbonyl-reactive group and a thiol-reactive group. In more particular embodiments, the thiol reactive group includes a maleimide group, the amine reactive group includes an active ester and the carbonyl-reactive group includes a hydrazine derivative.

In another aspect, methods for making the disclosed conjugates are provided. In one embodiment a method of making a conjugate includes forming a thiolated specific-binding moiety; reacting a nanoparticle having an amine group with a PEG maleimide/active ester bifunctional linker to form an activated nanoparticle; and reacting the thiolated specific-binding moiety with the activated signal-generating moiety to form the conjugate of the antibody and the signal-generating moiety. The thiolated specific-binding moiety can be formed by reduction of intrinsic cystine bridges of the specific-binding moiety using a reductant, or the thiolated specific-binding moiety can be formed by reacting the antibody with a reagent that introduces a thiol to the specific-binding moiety.

In another embodiment, a method for making a disclosed conjugate includes reacting a specific-binding moiety with an oxidant to form an aldehyde-bearing specific-binding moiety; reacting the aldehyde-bearing specific-binding moiety with a PEG maleimide/hydrazide bifunctional linker to form a thiol-reactive specific-binding moiety; and reacting the thiol-reactive specific-binding moiety with a thiolated nanoparticle to form the conjugate. In a particular embodiment, reacting the specific-binding moiety with an oxidant to form the aldehyde-bearing antibody includes oxidizing a glycosylated region of the specific-binding moiety (such as with periodate, I₂, Br₂, and combinations thereof) to form the aldehyde-bearing specific-binding moiety. The method can further include forming a thiolated nanoparticle from a nanoparticle, for example, by reacting a nanoparticle with a reagent that introduces a thiol group to the nanoparticle.

In another aspect, methods are disclosed for detecting molecules of interest in biological samples using disclosed conjugates, and in particular for multiplexed detection of molecules of interest using disclosed fluorescent nanoparticle conjugates. These and additional aspects, embodiments and features of the disclosure will become apparent from the detailed description and examples that follow.

### BRIEF DESCRIPTION OF THE DRAWINGS

FIG. 1 is series of images comparing fluorescence staining using a disclosed anti-biotin/QD605 conjugate in staining on CD20 versus a commercially available streptavidin/QD605 conjugate as a control.

FIG. 2 is a pair of images demonstrating multiplexed detection using disclosed conjugates in an IHC assay.

FIG. 3 is a series of images showing the high stability over time at elevated temperatures of a disclosed conjugate.

FIG. 4 is a series of images showing the results of an ISH assay using a disclosed conjugate.

FIG. 5 is a series of images showing the results of an IHC assay using a disclosed conjugate.

### DETAILED DESCRIPTION OF SEVERAL ILLUSTRATIVE EMBODIMENTS

Further aspects of the invention are illustrated by the following non-limiting examples, which proceed with respect to the abbreviations and terms defined below.

### I. ABBREVIATIONS

2-ME—2-mercaptoethanol

2-MEA—2-mercaptoethylamine

Ab—antibody

BSA—bovine serum albumin

DTE—dithioerythritol(cis-2,3-dihydroxy-1,4-dithiolbutane)

DTT—dithiothreitol(trans-2,3-dihydroxy-1,4-dithiolbutane)

FWHM—full-width half maximum

IHC—immunohistochemistry

ISH—in situ hybridization

MAL—maleimide

NHS—N-hydroxy-succinimide

NP—nanoparticle

PEG—polyethylene glycol

QD###—quantum dot (wavelength of fluorescence maximum)

SAMSA—S-Acetylmercaptosuccinic anhydride

SATA—N-succinimidyl S-acetylthioacetate

SATP—Succinimidyl acetyl-thiopropionate

SBM—Specific binding moiety

SMPT—Succinimidyloxycarbonyl-α-methyl-α-(2-pyridyldithio)toluene

SPDP—N-Succinimidyl 3-(2-pyridyldithio)propionate

TCEP—tris(carboxyethyl)phosphine

### II. TERMS

The terms “a,” “an” and “the” include both singular and plural referents unless the context clearly indicates otherwise.

The term “antibody” collectively refers to immunoglobulins or immunoglobulin-like molecules (including IgA, IgD, IgE, IgG and IgM, combinations thereof, and similar molecules produced during an immune response in any vertebrate, for example, in mammals such as humans, goats, rabbits and mice) and antibody fragments that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample. Antibody fragments include proteolytic antibody fragments [such as F(ab′)₂ fragments, Fab′ fragments, Fab′-SH fragments and Fab fragments as are known in the art], recombinant antibody fragments (such as sFv fragments, dsFv fragments, bispecific sFv fragments, bispecific dsFv fragments, diabodies, and triabodies as are known in the art), and camelid antibodies (see, for example, U.S. Pat. Nos. 6,015,695; 6,005,079-5,874,541; 5,840,526; 5,800,988; and 5,759,808).

The term “avidin” refers to any type of protein that specifically binds biotin to the substantial exclusion of other small molecules that might be present in a biological sample. Examples of avidin include avidins that are naturally present in egg white, oilseed protein (e.g., soybean meal), and grain (e.g., corn/maize) and streptavidin, which is a protein of bacterial origin.

The phrase “molecule of interest” refers to a molecule for which the presence, location and/or concentration is to be determined. Examples of molecules of interest include proteins and nucleic acid sequences tagged with haptens.

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

The term “quantum dot” refers to a nanoscale particle that exhibits size-dependent electronic and optical properties due to quantum confinement. Quantum dots have, for example, been constructed of semiconductor materials (e.g., cadmium selenide and lead sulfide) and from crystallites (grown via molecular beam epitaxy), etc. A variety of quantum dots having various surface chemistries and fluorescence characteristics are commercially available from Invitrogen Corporation, Eugene, Oreg. (see, for example, U.S. Pat. Nos. 6,815,064, 6,682,596 and 6,649,138, each of which patents is incorporated by reference herein). Quantum dots are also commercially available from Evident Technologies (Troy, N.Y.). Other quantum dots include alloy quantum dots such as ZnSSe, ZnSeTe, ZnSTe, CdSSe, CdSeTe, ScSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnCdSSe, ZnHgSSe, ZnCdSeTe, ZnHgSeTe, CdHgSSe, CdHgSeTe, InGaAs, GaAIAs, and InGaN quantum dots (Alloy quantum dots and methods for making the same are disclosed, for example, in US Application Publication No. 2005/0012182 and PCT Publication WO 2005/001889).

The term “specific-binding moiety” refers generally to a member of a specific-binding pair. Specific binding pairs are pairs of molecules that are characterized in that they bind each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Particular examples of specific binding moieties include specific binding proteins such as antibodies, lectins, avidins (such as streptavidin) and protein A. Specific binding moieties can also include the molecules (or portions thereof) that are specifically bound by such specific binding proteins.

### III. OVERVIEW

In one aspect, a specific-binding moiety/nanoparticle conjugate is disclosed that includes a specific-binding moiety covalently coupled to a nanoparticle through a heterobifunctional polyalkyleneglycol linker having the general structure show below: 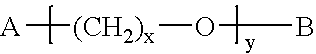
wherein A and B include different reactive groups, x is an integer from 2 to 10 (such as 2, 3 or 4), and y is an integer from 1 to 50, for example, an integer from 2 to 30 such as integer from 3 to 20 or an integer from 4 to 12. One or more hydrogen atoms in the formula can be substituted for functional groups such as hydroxyl groups, alkoxy groups (such as methoxy and ethoxy), halogen atoms (F, Cl, Br, I), sulfato groups and amino groups (including mono- and di-substituted amino groups such as dialkyl amino groups).

A and B can independently include a carbonyl-reactive group, an amine-reactive group, a thiol-reactive group or a photo-reactive group, but do not include the same reactive group. Examples of carbonyl-reactive groups include aldehyde- and ketone-reactive groups like hydrazine and hydrazide derivatives and amines. Examples of amine-reactive groups include active esters such as NHS or sulfo-NHS, isothiocyanates, isocyanates, acyl azides, sulfonyl chlorides, aldehydes, glyoxals, epoxides, oxiranes, carbonates, aryl halides, imidoesters, anhydrides and the like. Examples of thiol-reactive groups include non-polymerizable Michael acceptors, haloacetyl groups (such as iodoacetyl), alkyl halides, maleimides, aziridines, acryloyl groups, vinyl sulfones, benzoquinones, and disulfide groups such as pyridyl disulfide groups and thiols activated with Ellman's reagent. Examples of photo-reactive groups include aryl azide and halogenated aryl azides. Additional examples of each of these types of groups will be apparent to those skilled in the art. Further examples and information regarding reaction conditions and methods for exchanging one type of reactive group for another are provided in Hermanson, “Bioconjugate Techniques,” Academic Press, San Diego, 1996, which is incorporated by reference herein. In a particular embodiment, a thiol-reactive group is other than vinyl sulfone.

In some embodiments, a thiol-reactive group of the heterobifunctional linker is covalently attached to the specific-binding moiety and an amine-reactive group of the heterobifunctional linker is covalently attached to the nanoparticle, or vice versa. For example, a thiol-reactive group of the heterobifunctional linker can be covalently attached to a cysteine residue (such as following reduction of cystine bridges) of the specific-binding moiety or a thiol-reactive group of the heterobifunctional linker can be covalently attached to a thiol group that is introduced to the specific-binding moiety, and the amine-reactive group is attached to the nanoparticle.

Alternatively, an aldehyde-reactive group of the heterobifunctional linker can be covalently attached to the nanoparticle and an amine-reactive group of the heterobifunctional linker can be covalently attached to the nanoparticle, or vice versa. In a particular embodiment, an aldehyde-reactive group of the heterobifunctional linker can be covalently attached to an aldehyde formed on a glycosylated portion of a specific-binding moiety, and the amine-reactive group is attached to the nanoparticle.

In yet other embodiments, an aldehyde-reactive group of the heterobifunctional linker is covalently attached to the specific-binding moiety and a thiol-reactive group of the heterobifunctional linker is attached to the nanoparticle, or vice versa.

In some embodiments the heterobifunctional linker has the formula: 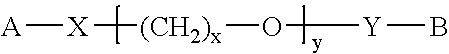
wherein A and B, which are different reactive groups as before; x and y are as before, and X and Y are spacer groups, for example, spacer groups having between 1 and 10 carbons such as between 1 and 6 carbons or between 1 and 4 carbons, and optionally containing one or more amide linkages, ether linkages, ester linkages and the like. Spacers X and Y can be the same or different, and can be straight-chained, branched or cyclic (for example, aliphatic or aromatic cyclic structures), and can be unsubstituted or substituted. Functional groups that can be substituents on a spacer include carbonyl groups, hydroxyl groups, halogen (F, Cl, Br and I) atoms, alkoxy groups (such as methoxy and ethoxy), nitro groups, and sulfate groups.

In particular embodiments, the heterobifunctional linker comprises a heterobifunctional polyethylene glycol linker having the formula: 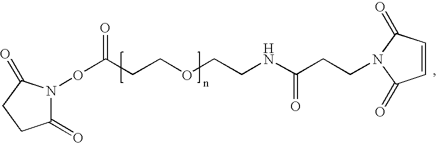
wherein n=1 to 50, for example, n=2 to 30 such as n=3 to 20 or n=4 to 12. In more particular embodiments, a carbonyl of a succinimide group of this linker is covalently attached to an amine group on the nanoparticle and a maleimide group of the linker is covalently attached to a thiol group of the specific-binding moiety, or vice versa. In other more particular embodiments, an average of between about 1 and about 10 specific-binding moieties are covalently attached to a nanoparticle. Examples of nanoparticles include semiconductor nanocrystals (such as quantum dots, obtained for example, from Invitrogen Corp., Eugene, Oreg.; see, for example, U.S. Pat. Nos. 6,815,064, 6,682,596 and 6,649,138, each of which patents is incorporated by reference herein), paramagnetic nanoparticles, metal nanoparticles, and superparamagnetic nanoparticles.

In other particular embodiments, the heterobifunctional linker comprises a heterobifunctional polyethylene glycol linker having the formula: 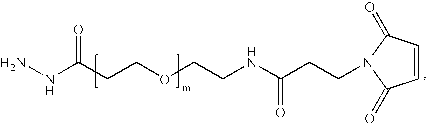
wherein m=1 to 50, for example, m=2 to 30 such as m=3 to 20 or m=4 to 12. In more particular embodiments, a hydrazide group of the linker is covalently linked with an aldehyde group of the specific-binding moiety and a maleimide group of the linker is covalently linked wtih a thiol group of the nanoparticle, or vice versa. In even more particular embodiments, the aldehyde group of the specific-binding moiety is an aldehyde group formed in an Fc portion of an antibody by oxidation of a glycosylated region of the Fc portion of the antibody. In other even more particular embodiments, an average of between about 1 and about 10 specific-binding moieties are covalently attached to the nanoparticle. Briefly, maleimide/hydrazide PEG-linkers of the formula above can be synthesized from corresponding maleimide/active ester PEG linkers (which are commercially available, for example, from Quanta Biodesign, Powell, Ohio) by treatment with a protected hydrazine derivative (such as a Boc-protected hydrazine) followed by treatment with acid.

In other particular embodiments, a heterobifunctional PEG-linked specific-binding moiety-nanoparticle conjugate comprises a conjugate having the formula: 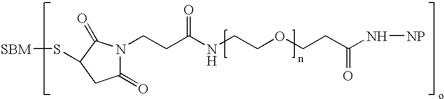
wherein SBM is a specific-binding moiety, NP is a nanoparticle, n=1 to 50 (such as n=2 to 30, n=3 to 20 or n=4 to 12) and o=1 to 10 (such as o=2 to 6 or o=3 to 4); or 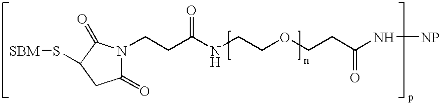
wherein SBM is a specific-binding moiety, NP is a nanoparticle, n=1 to 50 (such as n=2 to 30, n=3 to 20 or n=4 to 12) and p=1 to 10 (such as p=2 to 6 or p=3 to 4).

In yet other particular embodiments, a heterobifunctional PEG-linked specific-binding moiety-nanoparticle conjugate comprises a conjugate having the formula: 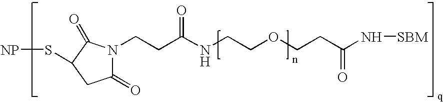
wherein SBM is a specific-binding moiety, NP is a nanoparticle, n=1 to 50 (such as n=2 to 30, n=3 to 20 or n=4 to 12) and q=1 to 10 (such as q=2 to 6 or q=3 to 4); or 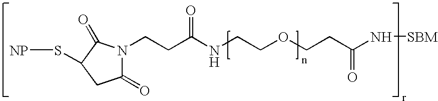
wherein SBM is a specific-binding moiety, NP is a nanoparticle and n=1 to 50 (such as n=2 to 30, n=2 to 20 or n=4 to 12) and r=to 10 (such as r=2 to 6 or r=3 to 4).

In still other particular embodiments, a heterobifunctional PEG-linked specific-binding moiety-nanoparticle conjugate comprises a conjugate having the formula: 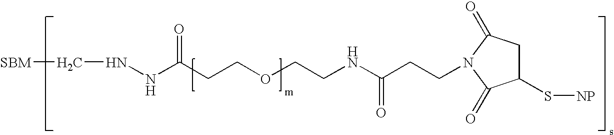
wherein SBM is a specific-binding moiety, NP is a nanoparticle, m=1 to 50 (such as m=2 to 30, m=3 to 20 or m=4 to 12) and s=1 to 10 (such as s=2 to 6 or s=3 to 4); or 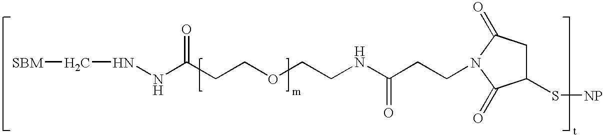
wherein SBM is a specific-binding moiety, NP is a nanoparticle, m=1 to 50 (such as m=2 to 30, 2 to 20 or 4 to 12) and t=1 to 10 (such as t=2 to 6 or t=3 to 4).

In still further particular embodiments, a heterobifunctional PEG-linked specific-binding moiety-nanoparticle conjugate comprises a conjugate having the formula: 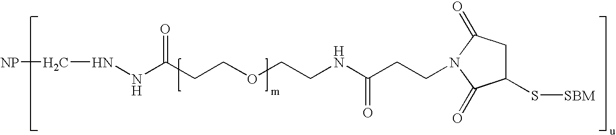
wherein SBM is a specific-binding moiety, NP is a nanoparticle, m=1 to 50 (such as m=2 to 30, m=3 to 20 or m=4 to 12) and u=1 to 10 (such as u=2 to 6 or u=3 to 4); or 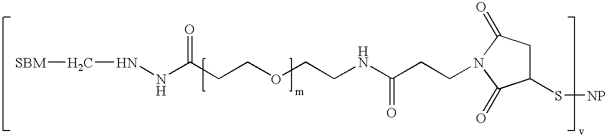
wherein SBM is a specific-binding moiety, NP is a nanoparticle, m=1 to 50 (such as m=2 to 30, m=2 to 20 or m=4 to 12) and v=1 to 10 (such as v=2 to 6 or v=3 to 4).

The SBM in these conjugates can include, for example, an antibody, a nucleic acid, a lectin or an avidin such as streptavidin. If the SBM includes an antibody, the antibody can specifically bind any particular molecule or particular group of highly similar molecules, and in particular embodiments, the antibody comprises an anti-hapten antibody (which can, for example, be used to detect a hapten-labeled probe sequence directed to a nucleic acid sequence of interest) or an antibody that specifically binds a particular protein that may be present in a sample. Haptens are small organic molecules that are specifically bound by antibodies, although by themselves they will not elicit an immune response in an animal and must first be attached to a larger carrier molecule such as a protein to stimulate an immune response. Examples of haptens include di-nitrophenol, biotin, and digoxigenin. In still other particular embodiments, the antibody comprises an anti-antibody antibody that can be used as a secondary antibody in an immunoassay. For example, the antibody can comprise an anti-IgG antibody such as an anti-mouse IgG antibody, an anti-rabbit IgG antibody or an anti-goat IgG antibody.

Disclosed conjugates can be utilized for detecting molecules of interest in any type of binding immunoassay, including immunohistochemical binding assays and in situ hybridization methods employing immunochemical detection of nucleic acid probes. In one embodiment, the disclosed conjugates are used as a labeled primary antibody in an immunoassay, for example, a primary antibody directed to a particular molecule or a hapten-labeled molecule. Or, where the molecule of interest is multi-epitopic a mixture of conjugates directed to the multiple epitopes can be used. In another embodiment, the disclosed conjugates are used as secondary antibodies in an immunoassay (for example, directed to a primary antibody that binds the molecule of interest; the molecule of interest can be bound by two primary antibodies in a sandwich-type assay when multi-epitopic). In yet another embodiment, mixtures of disclosed conjugates are used to provide further amplification of a signal due to a molecule of interest bound by a primary antibody (the molecule of interest can be bound by two primary antibodies in a sandwich-type assay). For example, a first conjugate in a mixture is directed to a primary antibody that binds a molecule of interest and a second conjugate is directed to the antibody portion of the first conjugate, thereby localizing more signal-generating moieties at the site of the molecule of interest. Other types of assays in which the disclosed conjugates can be used are readily apparent to those skilled in the art.

In another aspect, a method is disclosed for preparing a specific-binding moiety-nanoparticle conjugate, the method including forming a thiolated specific-binding moiety from a specific-binding moiety; reacting a nanoparticle having an amine group with a PEG maleimide/active ester bifunctional linker to form an activated nanoparticle; and reacting the thiolated specific-binding moiety with the activated nanoparticle to form the specific-binding moiety-nanoparticle conjugate.

A thiolated specific-binding moiety can be formed by reacting the specific-binding moiety with a reducing agent to form the thiolated specific-binding moiety, for example, by reacting the specific-binding moiety with a reducing agent to form a thiolated specific-binding moiety having an average number of thiols per specific-binding moiety of between about 1 and about 10. The average number of thiols per specific-binding moiety can be determined by titration. Examples of reducing agents include reducing agents selected from the group consisting of 2-mercaptoethanol, 2-mercaptoethylamine, DTT, DTE and TCEP, and combinations thereof. In a particular embodiment the reducing agent is selected from the group consisting of DTT and DTE, and combinations thereof, and used at a concentration of between about 1 mM and about 40 mM.

Alternatively, forming the thiolated specific-binding moiety includes introducing a thiol group to the specific-binding moiety. For example, the thiol group can be introduced to the specific-binding moiety by reaction with a reagent selected from the group consisting of 2-Iminothiolane, SATA, SATP, SPDP, N-Acetylhomocysteinethiolactone, SAMSA, and cystamine, and combinations thereof (see, for example, Hermanson, “Bioconjugate Techniques,” Academic Press, San Diego, 1996, which is incorporated by reference herein). In a more particular embodiment, introducing the thiol group to the specific-binding moiety includes reacting the specific-binding moiety with an oxidant (such as periodate) to convert a sugar moiety (such as in a glycosylated portion of an antibody) of the specific-binding moiety into an aldehyde group and then reacting the aldehyde group with cystamine. In another more particular embodiment, the specific binding moiety includes streptavidin and introducing the thiol group comprises reacting the streptavidin with 2-iminothiolane (Traut reagent).

In other particular embodiments, reacting the nanoparticle with a PEG maleimide/active ester bifunctional linker to form an activated nanoparticle includes reacting the nanoparticle with a PEG maleimide/active ester having the formula: 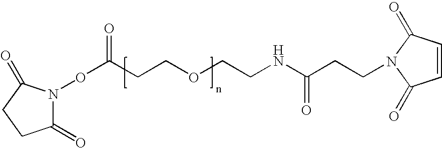
wherein n=1 to 50, for example, n=2 to 30 such as n=3 to 20 or n=4 to 12.

In a further aspect, a method is disclosed for preparing a specific-binding moiety-nanoparticle conjugate composition that includes reacting a specific-binding moiety with an oxidant to form an aldehyde-bearing specific-binding moiety; reacting the aldehyde-bearing specific-binding moiety with a PEG maleimide/hydrazide bifunctional linker to form a thiol-reactive specific-binding moiety; and reacting the thiol-reactive specific-binding moiety with a thiolated nanoparticle to form the specific-binding moiety-nanoparticle conjugate. In a particular embodiment, the specific-binding moiety is an antibody and reacting the specific-binding moiety with an oxidant to form the aldehyde-bearing specific-binding moiety includes oxidizing (such as with periodate, I₂, Br₂, or a combination thereof, or neuramidase/galactose oxidase) a glycosylated region of the antibody to form the aldehyde-bearing antibody. In a more particular embodiment, reacting an antibody with an oxidant to form an aldehyde-bearing antibody includes introducing an average of between about 1 and about 10 aldehyde groups per antibody.

A thiolated nanoparticle also can be formed from a nanoparticle by introducing a thiol group to the nanoparticle (for example, by reacting a nanoparticle with a reagent selected from the group consisting of 2-Iminothiolane, SATA, SATP, SPDP, N-Acetylhomocysteinethiolactone, SAMSA, and cystamine, and combinations thereof).

In particular embodiments, the PEG maleimide/hydrazide bifunctional linker has the formula: 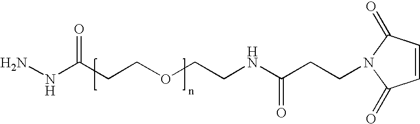
wherein m=1 to 50, for example, m=2 to 30 such as m=3 to 20 or m=4 to 12.

In a still further aspect, a method is disclosed for detecting a molecule of interest in a biological sample that includes contacting the biological sample with a heterobifunctional PEG-linked specific-binding moiety-nanoparticle conjugate and detecting a signal generated by the specific-binding moiety-nanoparticle conjugate. The biological sample can be any sample containing biomolecules (such as proteins, nucleic acids, lipids, hormones etc.), but in particular embodiments, the biological sample includes a tissue section (such as obtained by biopsy) or a cytology sample (such as a Pap smear or blood smear). In a particular embodiment, the heterobifunctional PEG-linked specific-binding moiety-nanoparticle conjugate includes a specific-binding moiety covalently linked to a quantum dot.

### IV. Examples

The following non-limiting examples are provided to further illustrate certain aspects of the invention.

A. Preparation of Specific-Binding Moiety-Nanoparticle Conjugates Using Maleimide PEG Active Esters.

In one embodiment, a disclosed specific-binding moiety nanoparticle conjugate is prepared according to the processes described in schemes 1 to 3 below, wherein the heterobifunctional polyalkylene glycol linker is a polyethylene glycol linker having an amine-reactive group (active ester) and a thiol-reactive group (maleimide). As shown in Scheme 1, a nanoparticle (such as a quantum dot) that has one or more available amine groups is reacted with an excess of the linker to form an activated nanoparticle.

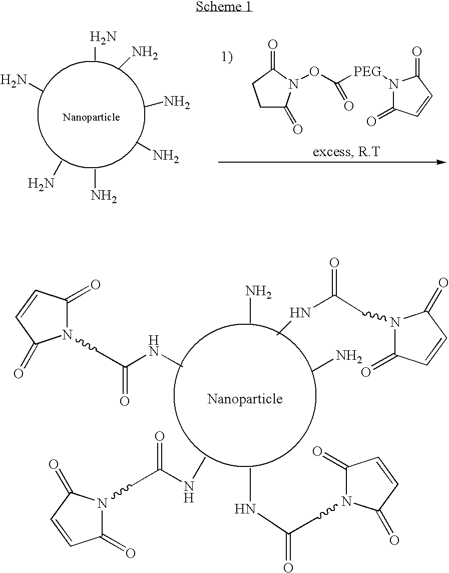

Thiol groups are introduced to the antibody by treating the antibody with a reducing agent such as DTT as shown in Scheme 2. For a mild reducing agent such as DTE or DTT, a concentration of between about 1 mM and about 40 mM, for example, a concentration of between about 5 mM and about 30 mM such as between about 15 mM and about 25 mM, is utilized to introduce a limited number of thiols (such as between about 2 and about 6) to the antibody while keeping the antibody intact (which can be determined by size-exclusion chromatography). A suitable amount of time for the reaction with a solution of a particular concentration can be readily determined by titrating the number of thiols produced in a given amount of time, but the reaction is typically allowed to proceed from 10 minutes to about one day, for example, for between about 15 minutes and about 2 hours, for example between about 20 minutes and about 60 minutes.

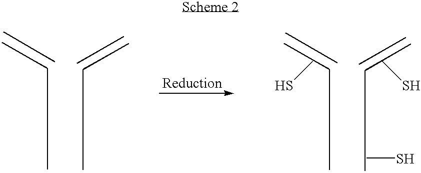

The components produced according to Schemes 1 and 2 are then combined to give a conjugate as shown in Scheme 3.

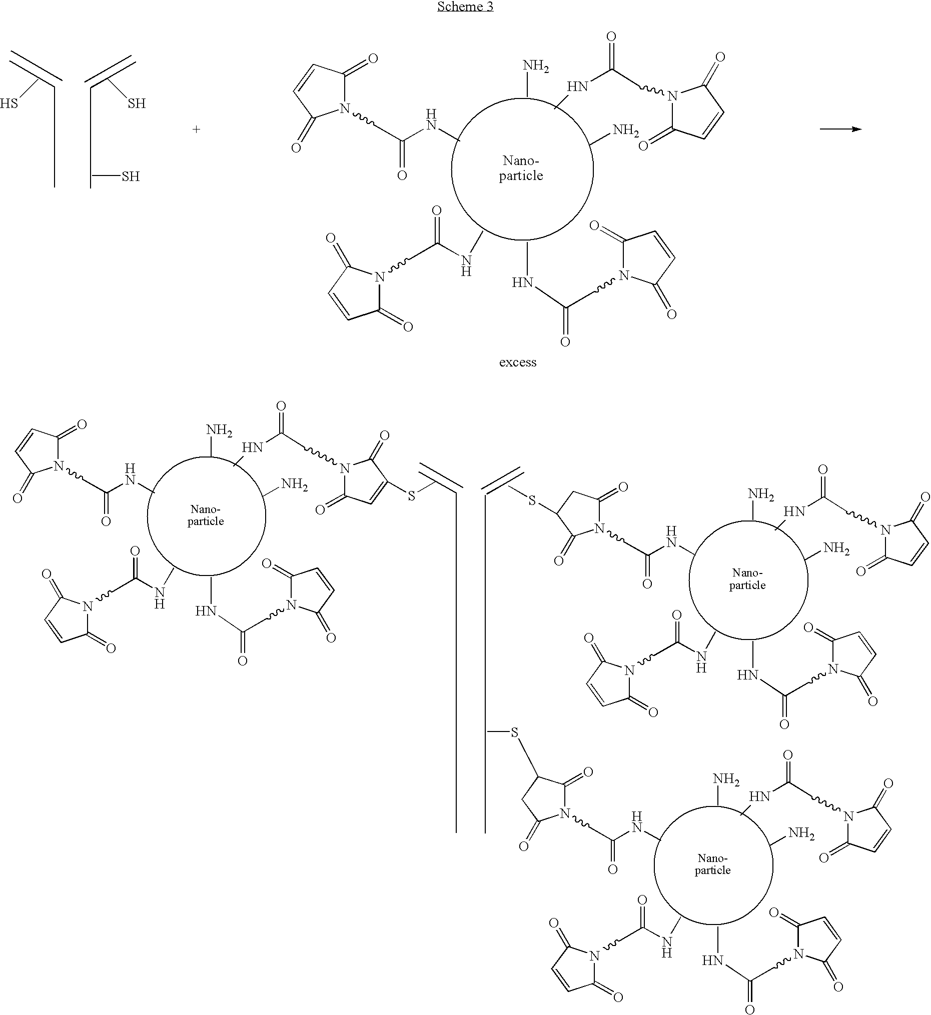

Although Schemes 1-3 illustrate an optimal process for maleimide PEG active esters, wherein the nanoparticle is first activated by reacting an amine group(s) with the active ester of the linker to form an activated nanoparticle, it is also possible to first activate the antibody by reacting either an amine(s) or a thiol(s) on the antibody with the linker and then react the activated antibody with the nanoparticle [having either a thiol(s) or an amine(s) to react with the remaining reactive group on the linker as appropriate].

Thus, in an alternative embodiment, an antibody is activated for conjugation and then conjugated to a nanoparticle as shown in Schemes 4 and 5 below. In Scheme 4, the antibody is activated instead of the nanoparticle as was shown in Scheme 1. In the particular embodiment of scheme 4, a sugar moiety (such as located in a glycosylated region of the Fc portion of the antibody) is first oxidized to provide an aldehyde group, which is then reacted with an aldehyde-reactive group of the linker (such as a hydrazide group of the illustrated maleimide/hydrazide PEG linker).

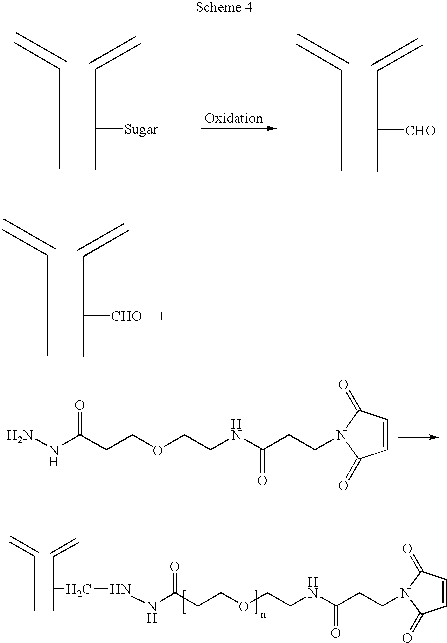

Then, as shown in Scheme 5, a thiol-reactive group of the linker portion of the activated antibody (such as a maleimide group as illustrated) is then reacted with a thiol group on the nanoparticle. Again, the process can be reversed, wherein the linker is first reacted with an aldehyde group on the nanoparticle (formed, for example, by oxidation of a sugar moiety) to form an activated nanoparticle, and then the activated nanoparticle can be reacted with a thiol group on the antibody.

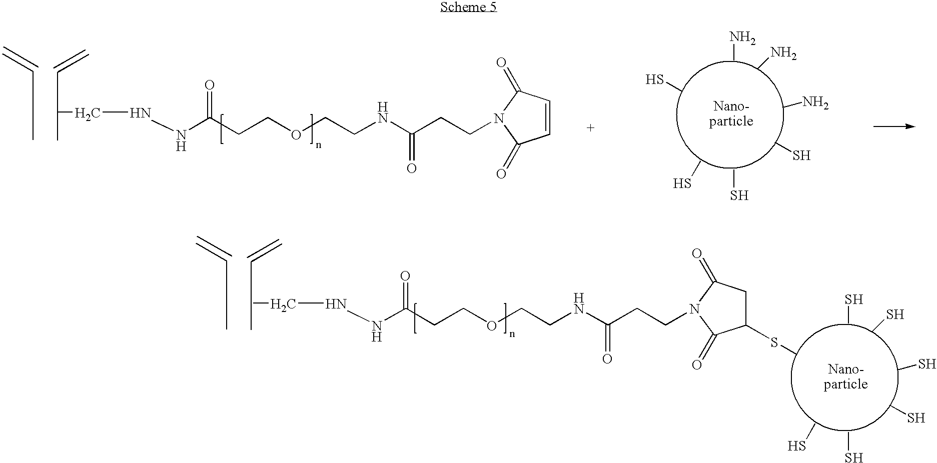

Although schemes 1-5 above and 6 that follows show particular examples of conjugates for illustrative purposes, it is to be understood that the ratio of specific-binding moiety (in this case, antibody) to nanoparticle in the disclosed conjugates can vary from multiple (such as 5, 10, 20 or more) specific binding moieties per nanoparticle to multiple nanoparticles per specific-binding moiety (such as 5, 10, 20 or more).

### Example B

### Introduction of Thiols to Antibodies

To activate an antibody for conjugation, for example, an anti-mouse IgG or anti-rabbit IgG antibody, the antibody can be incubated with 25 mmol DTT at ambient temperature (23-25° C.) for about 25 minutes. After purification across a PD-10 SE column, DTT-free antibody, typically with two to six free thiols, is obtained (Scheme 2). The exemplary procedure outlined for preparing goat anti-mouse IgG thiol is generally applicable to other antibodies. The number of thiols per antibody can be determined by titration, for example, by using the thiol assay described in U.S. Provisional Patent Application No. 60/675,759, filed Apr. 28, 2005, which application is incorporated by reference herein.

### Example C

### Conjugates of Immunoglobulins and Streptavidin with CdSe/ZnS Quantum Dots for Ultrasensitive (and Multiplexed) Immunohistochemical and In Situ Hybridization Detection in Tissue Samples

Semiconductor nanocrystals, often referred to as quantum dots, can be used in biological detection assays for their size-dependent optical properties. Quantum dots offer the ability to exhibit bright fluorescence as a result of high absortivities and high quantum yields in comparison to typical organic fluorphores. Additionally, the emission is tunable and stable to photobleaching, allowing for archivability. For detection and assay purposes, these robust fluorophores provide advantages in multiplexing assays. For example, excitation for these visible/NIR emitters is possible with a single source. However, a limiting factors in biological imaging is the sensitivity and stability of bioconjugates. In order to effectively utilize quantum dots in multicolor assays, each dot is desirably specific and sensitive.

A method of incorporating an immunoglobulin into a quantum dot shell is described int this example. This method relies on 1.) Functionalization of amine-terminated quantum dot capping groups with a suitable NHS ester-(PEG)x-maleimide, (x=4,8,12) heterobifunctional 2.) Reduction of native disulfides throughout immunoglobulins via time-dependent treatment with dithiothreitol (DTT) 3.) Derivatizing maleimide-terminated quantum dots with these thiolated immunoglobulins 4.) Purifying the conjugates with size-exclusion chromatography. The process is depicted in Scheme 6.

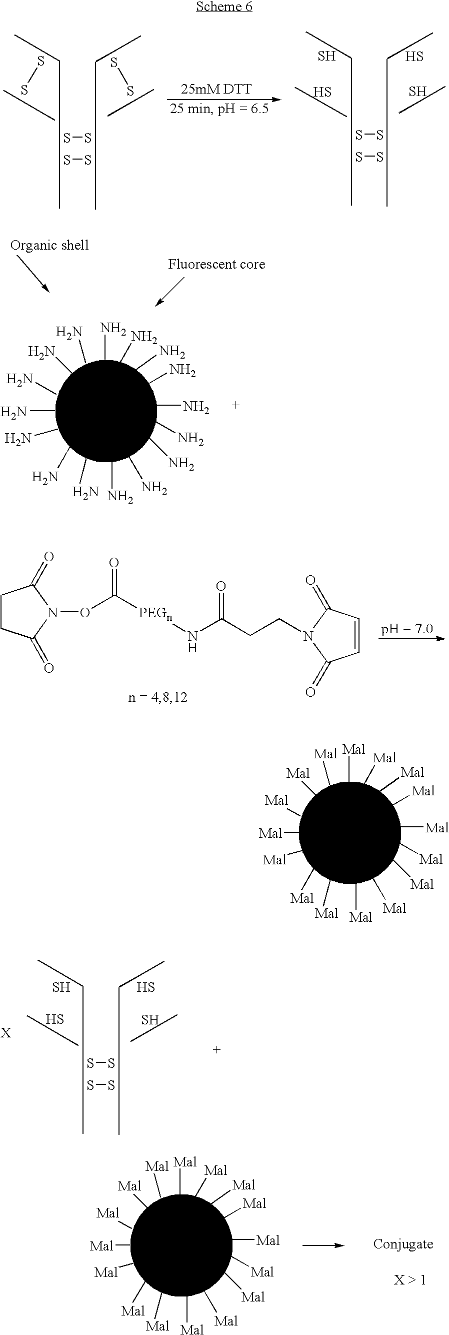

A streptavidin conjugate can be made by substituting a thiolated streptavidin for the thiolated immunoglobulin in the process. For example, a streptavidin molecule treated with 2-iminothiolane.

The quantum dots used in this example were protected by an electrostatically bound shell of trioctyl phosphine oxide (TOPO) and an intercalating amphiphilic polymer to induce water solubility. This polymer has approximately 30 terminal amine groups for further functionalization. See E. W. Williams, et. al. “Surface-Modified Semiconductive and Metallic Nanoparticles Having Enhanced Dispersibility in Aqueous Media”, U.S. Pat. No. 6,649,138 (incorporated by reference, herein). In order to form highly sensitive quantum dot conjugates, antibodies were attached to the quantum dots with varying ratios. The chemistry is similar to that described in U.S. Provisional Patent Application No. 60/675,759, filed Apr. 28, 2005, which is incorporated by reference herein.

This methodology is advantageous due to the need for few reagents because native disulfides are used. Additionally, the antibody remains discrete and does not form fragments. This allows for two binding sites from each tethered antibody. Furthermore, highly stable and bright conjugates are produced. The brightness surpasses commercially available streptavidin-QD conjugates (Invitrogen Corporation, Eugene, Oreg.) on the same tissue. Goat anti-biotin and rabbit anti-DNP antibodies conjugated to quantum dots of differing wavelengths of emission were produced, thereby permitting multiplex assays. HPV detection through FISH was demonstrated with the disclosed quantum dot conjugates.

Materials

DTT was purchased from Aldrich and quantum dots were purchased from Quantum Dot, Co. and used as received. NHS-dPEG₁₂-MAL and NHS-dPEG₄-MAL were purchased from Quanta Biodesign. Goat anti-biotin was received lyophilized from Sigma and rabbit anti-DNP was received at 2 mg/mL in buffer at pH=7.2 from Molecular Probes. Antibody concentrations were calculated using ε₂₈₀=1.4 ml mg⁻¹ cm⁻¹. Immunopure streptavidin was received from Pierce. Streptavidin concentrations were determined using ε₂₈₀=3.4 ml mg⁻¹ cm⁻¹. Quantum dot concentrations were determined using ε_{601(±}3)=650 000 M⁻¹ cm⁻¹ for 605 nm emitting quantum dots (QD₆₀₅) and ε_{645(±}3)=700 000 M⁻¹ cm⁻¹ for QD 655. Deionized water was passed through a Milli-Q Biocel System to reach a resistance of 18.2 MΩ. Buffer exchange was performed on PD-10 columns (GE Biosciences). Size-exclusion chromatography (SEC) was performed on Akta purifiers (GE Biosciences) which was calibrated to protein standards of known molecular weight. The flow rate was 0.9 mL/min on a Superdex 200 GL 10/300 (GE Biosciences).

Reduction of Inter-Chain Disulfides on Antibodies

To a solution of polyclonal antibiotin, which was received lyophilized and was reconstituted to 3.0 mg/ml in 0.1 M Na phosphate, 0.1 M EDTA, pH=6.5 buffer was added DTT at a final concentration of 25 mM. This was done on scales from 0.67 ml to 2.7 ml. This mixture was rotated for precisely 25 minutes before eluting on a PD-10 in 0.1 M Na phosphate, 0.1 M NaCl, pH=7.0 buffer. The same procedure was repeated for anti-DNP, although this was received in buffer as 2 mg/mL. The number of antibodies incorporated was approximately equal

Thiolation of Streptavidin

Traut's solution was prepared, which consisted of 0.275 mg/mL 2-iminothiolane in 0.15 M NaCl, 1 mM EDTA, 50 M triethanolamine HCl, pH=8.0 buffer. To 0.5 mL of a solution of streptavidin (4.1 mg/mL) in 0.1 M Na phosphate, 0.1 M NaCl, pH=7.0 buffer was added 0.25 mL Traut's solution and rotated for 45 minutes.

Synthesis of QD-dPEGₓ-MAL

To a solution of quantum dots (8-9 uM) in borate buffer, pH=8.0) was added 60 fold excess of NHS-dPEGₓ-MAL (x=4,12) and rotated for 2 hours. The quantum dots were purified via PD-10 chromatography in 0.1 M Na phosphate, 0.1 M NaCl, pH=7.0 buffer.

Synthesis of QD-MAL-Antibody Conjugate

The purified QD-maleimide was combined with the purified thiolated antibody in molar ratios of 2:1, 5:1, and 10:1 antibodies/QD and rotated for a 16 hour period. SEC was performed in 1×PBS buffer, pH=7.5.

Synthesis of QD-MAL-Streptavidin Conjugate

The purified QD-maleimide was combined with the thiolated streptavidin in a molar ratio of 5:1 proteins/QD and rotated for a 16 hour period. SEC was performed in 1×PBS buffer, pH=7.5.

Evaluation of QD-MAL Conjugates Using Biotinylated Microtiter Plates

Biotinylated plates were purchased from Pierce Biotechnology. Staining was performed in triplicate at 40 nM or with serial titrations. These were performed in PBS pH=7.5 buffer.

Tissue Staining Details

IHC—Staining was performed with 40 nM and 20 nM solutions of quantum dot conjugates in casein. This was carried out on a Ventana Benchmark Instrument (VMSI, Tucson, Ariz.): The tissue sample was deparaffinized and the epitope-specific antibody was applied. After incubation for 32 minutes, the universal secondary antibody (biotinylated) was added. Incubation again occurred for 32 minutes. The anti-biotin quantum dot conjugates (100 uL) were then applied manually and also incubated for 32 minutes. When used, a DAPI counterstain was applied, followed by an 8 minute incubation. The slide was treated to a detergent wash, dehydrated with ethanol and xylene, and coverslipped before viewing with fluorescence microscopy.

ISH—Staining was performed with 40 nM solutions of quantum dots in casein. Again, this was carried out on a Ventana Benchmark Instrument. The paraffin coated tissue was warmed to 75° C., incubated for 4 minutes, and treated twice with EZPrep™ volume adjust (VMSI). The second treatment was followed with a liquid coverslip, a 4 minute incubation at 76° C., and a rinse step to deparaffin the tissue. Cell conditioner #2 (VMSI) was added and the slide was warmed to 90° C. for 8 minutes. Cell conditioner #2 was added again for another incubation at 90° C. for 12 minutes. The slide was rinsed with reaction buffer (VMSI), cooled to 37° C., and ISH-Protease 3 (100 μL, VMSI) was added. After 4 minutes, iView™+HybReady™ (100 μL, VMSI) was applied and also incubated for 4 minutes. HPV HR Probe (200 μL, VMSI) was added and incubated for 4 minutes at 37° C., followed by 12 minutes at 95° C. and 124 minutes at 52° C. The slide was rinsed and warmed again to 72° C. for 8 minutes two separate times.

Anti-Biotin Quantum Dot Conjugates

At 37° C., the primary antibody, iView+Rabbit Anti-DNP (100 μL, VMSI), was applied and incubated for 20 minutes. For amplification, iView+Amp (100 μL, VMSI), was applied and incubated for 8 minutes. The secondary antibody, which is Goat Anti-Mouse Biotin, iVIEW+Biotin−Ig (100 μL, VMSI) was applied and incubated for 12 minutes. Finally 100 uL of the quantum dot/antibody conjugate was applied, incubated for 28 minutes, and rinsed. The slide was rinsed with reaction buffer, dehydrated with ethanol and xylene, followed by addition of the cover slip.

Anti-DNP Quantum Dots

At 37° C., QD/Anti-DNP conjugates were applied (100 μL), incubated for 28 minutes, and rinsed. Again the slide was rinsed and coverslipped.

Fluorescence Microscopy

Imaging was performed on a Nikon fluorescence scope. Unmixing of fluorescence spectra was achieved utilizing a CRi camera. DAPI was used for counterstaining for multiplexing.

Comparison to QD-SA Conjugates

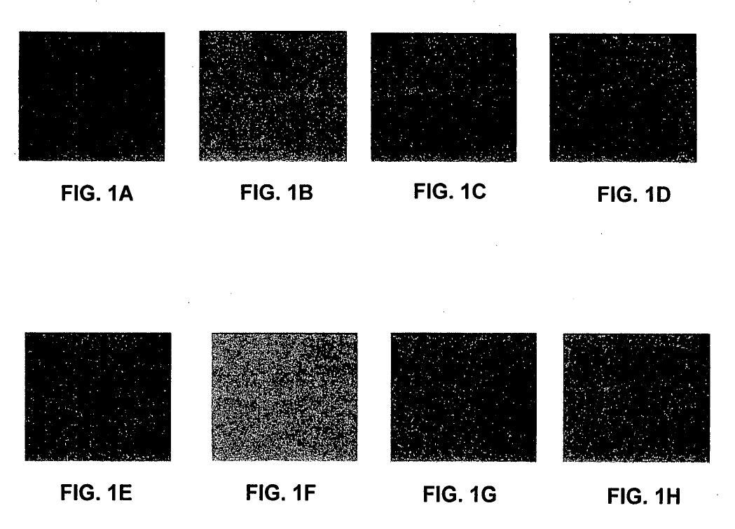

FIG. 1 compares an anti-biotin/QD605 conjugate in staining on CD20 versus a commercially available streptavidin/QD605 conjugate as a control. FIGS. 1A to 1D show, respectively, staining with 40 mM solutions of a commercially available streptavidin/QD605, 2:1 AB/QD 605, 5:1 AB/QD 605, 10:1 AB/QD605. Likewise, FIGS. 1E to 1H show staining with 20 nM solutions of commercially available streptavidin/QD605, 2:1 AB/QD 605, 5:1 AB/QD 605, 10:1 AB/QD605.

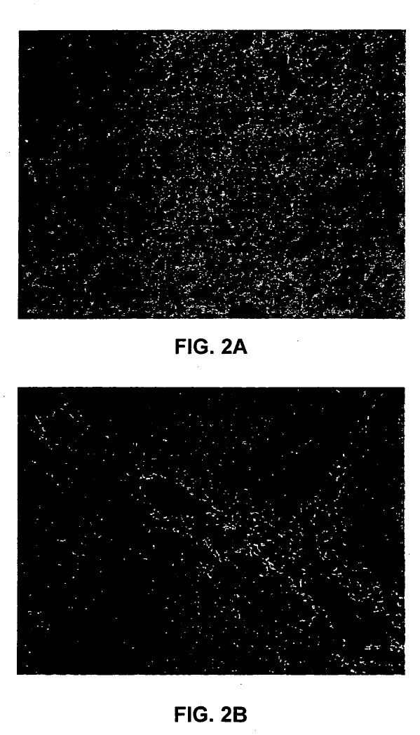

FIG. 2 demostrates multiplex use of the disclosed conjugates. Specifically, multiplexing with a QD605 conjugate, a QD655 conjugate, and DAPI counterstain (blue). FIG. 2A shows staining of neurofilament with a QD605 (Green) conjugate and GFAP staining with a QD655 (Red) conjugate. FIG. 2B shows staining of cadherin with a QD655 (Red) conjugate and staining of CD20 with a QD605 (Green) conjugate.

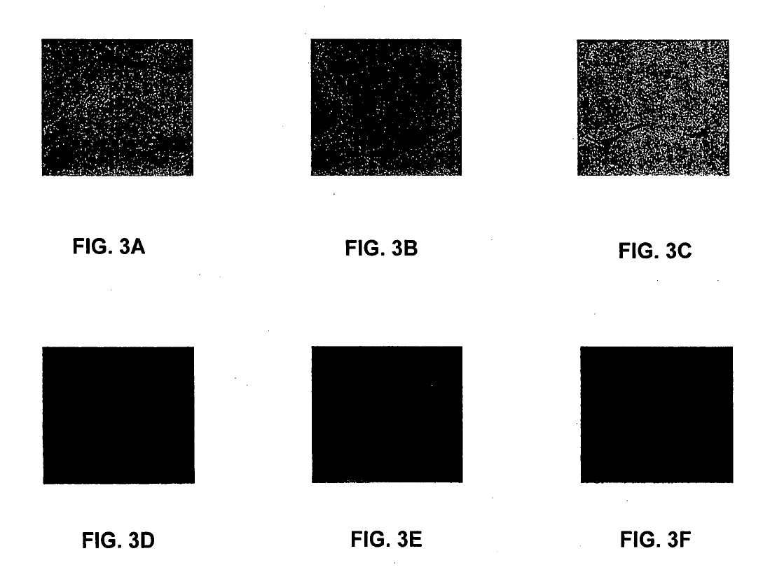

FIG. 3 demonstrates the stability of the disclosed conjugates, thereby also demonstrating the archivability of samples stained with the disclosed conjugates. The stability at 45° C. of a QD605 conjugate and a QD655 conjugate was examined by staining CD20 on tonsil tissue sections.

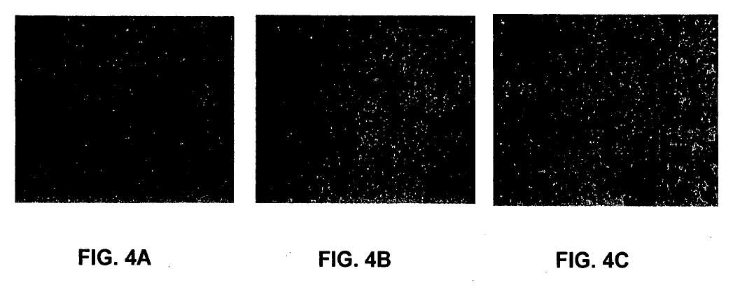

FIG. 4 demonstrates the use of disclosed conjugates for an ISH assay for human papilloma virus (HPV) using an HPV probe and 1:5 QD/Ab conjugates. FIGS. 4A to 4C, respectively, show staining with QD655/antibiotin-Ab conjugate, QD605/antibiotin-Ab conjugate, and QD605/antiDNP conjugate.

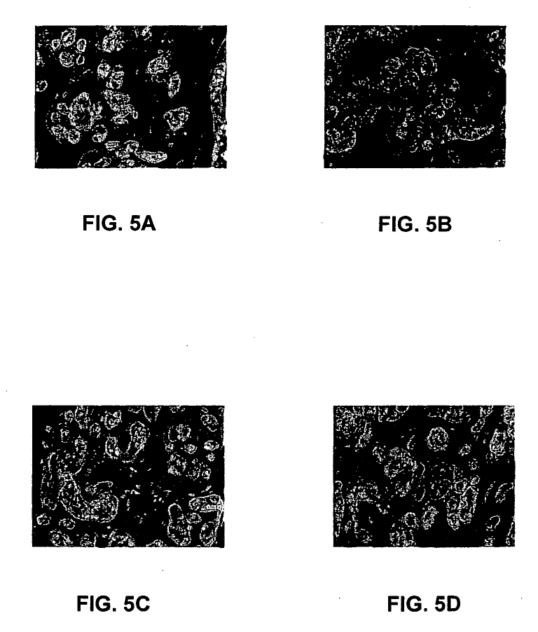

FIG. 5 demonstrates the use in an IHC assay of streptavidin-QD conjugates according to the disclosure. In particular FIGS. 5A to 5D show staining of CD34 in placental tissue using, respectively, 5, 10, 20, and 40 nM concentrations of a streptavidin/QD605 conjugate according to the disclosure.

Although the principles of the present invention are described with reference to several embodiments, it should be apparent to those of ordinary skill in the art that the details of the embodiments may be modified without departing from such principles. The present invention includes all modifications, variations, and equivalents thereof as fall within the scope and spirit of the following claims.

We claim:

1. A conjugate comprising a specific-binding moiety and a nanoparticle covalently coupled through a heterobifunctional PEG linker.

2. The conjugate of claim 1, wherein a thiol-reactive group of the linker is covalently attached to the specific-binding moiety and an amine-reactive group of the linker is covalently attached to the nanoparticle.

3. The conjugate of claim 2, wherein the thiol-reactive group of the linker is covalently attached to a cysteine residue of the specific-binding moiety.

4. The conjugate of claim 2, wherein the thiol-reactive group of the linker is covalently attached to a thiol group that is introduced to the specific-binding moiety.

5. The conjugate of claim 1, wherein an aldehyde-reactive group of the linker is covalently attached to the specific-binding moiety and an amine-reactive group of the linker is covalently attached to the nanoparticle.

6. The conjugate of claim 5, wherein the aldehyde-reactive group of the linker is covalently attached to an aldehyde formed on a glycosylated portion of the specific-binding moiety.

7. The conjugate of claim 1, wherein an aldehyde-reactive group of the linker is covalently attached a specific-binding moiety and a thiol-reactive group of the heterobifunctional linker is attached to the nanoparticle.

8. The conjugate of claim 1, wherein the heterobifunctional linker comprises a heterobifunctional polyethylene glycol linker having the formula: 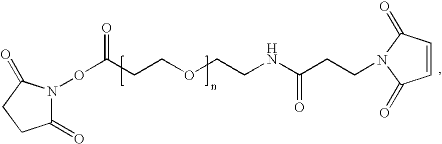
wherein n=1 to 50; or 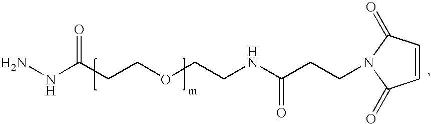
wherein m is from 1 to 50.

9. The conjugate of claim 8, wherein the conjugate has the formula: 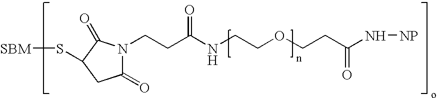
wherein SBM is a specific-binding moiety, NP is a nanoparticle, n=1 to 50 and o=1 to 10.

10. The conjugate of claim 8, wherein the conjugate has the formula: 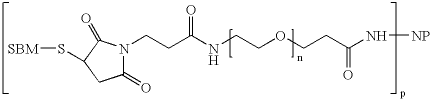
wherein SBM is a specific-binding moiety, NP is a nanoparticle, n=1 to 50 and p=1 to 10.

11. The conjugate of claim 8, wherein the conjugate has the formula: 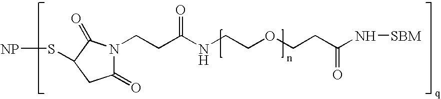
wherein SBM is a specific-binding moiety, NP is a nanoparticle, n=1 to 50 and q=1 to 10.

12. The conjugate of claim 8, wherein the conjugate has the formula: 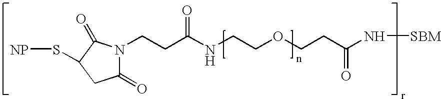
wherein SBM is a specific-binding moiety, NP is a nanoparticle and n=1 to 50 and r=1 to 10.

13. The conjugate of claim 8, wherein the conjugate has the formula: 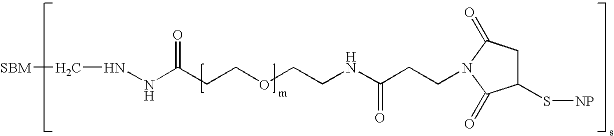
wherein SBM is a specific-binding moiety, NP is a nanoparticle, m=1 to 50 and s=1 to 10.

14. The conjugate of claim 8, wherein the conjugate has the formula: 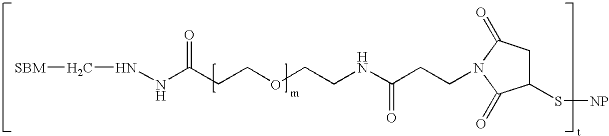
wherein SBM is a specific-binding moiety, NP is a nanoparticle, m=1 to 50 and t=1 to 10.

15. The conjugate of claim 8, wherein the conjugate has the formula: 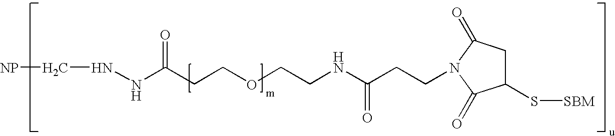
wherein SBM is a specific-binding moiety, NP is a nanoparticle, m=1 to 50 and u=1 to 10.

16. The conjugate of claim 8, wherein the conjugate has the formula: 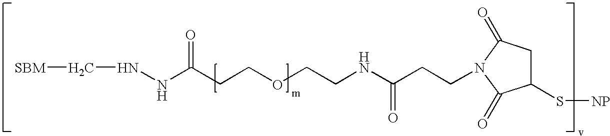
wherein SBM is a specific-binding moiety, NP is a nanoparticle, m=1 to 50 and v=1 to 10.

17. The conjugate of claim 1, wherein the nanoparticle comprises a quantum dot.

18. The conjugate of claim 1, wherein the specific-binding moiety comprises an antibody.

19. The conjugate of claim 1, wherein the specific-binding moiety comprises an avidin.

20. A method for preparing a specific-binding moiety-nanoparticle conjugate composition, comprising:
forming a thiolated specific-binding moiety from a specific-binding moiety;
reacting an amine group of a nanoparticle with a maleimide/active ester bifunctional PEG linker to form an activated nanoparticle; and
reacting the thioated specific-binding moiety with the activated nanoparticle to form the specific-binding moiety-nanoparticle conjugate.

21. The method of claim 20, wherein forming the thiolated specific-binding moiety comprises reacting the specific-binding moiety with a reducing agent to form the thiolated specific-binding moiety.

22. The method of claim 20, wherein the specific-binding moiety comprises an antibody and forming the thiolated specific-binding moiety comprises reacting the antibody with a reducing agent to form a thiolated antibody.

23. The method of claim 22, wherein reacting the antibody with the reducing agent to form the thiolated antibody comprises forming an antibody with an average number of thiols per antibody of between about 1 and about 10.

24. The method of claim 22, wherein reacting the antibody with the reducing agent comprises reacting the antibody with a reducing agent selected from the group consisting of 2-mercaptoethanol, 2-mercaptoethylamine, DTT, DTE and TCEP, and combinations thereof.

25. The method of claim 24, wherein reacting the antibody with the reducing agent comprises reacting the antibody with a reducing agent selected from the group consisting of DTT and DTE, and combinations thereof.

26. The method of claim 25, wherein reacting the antibody with the reducing agent comprises reacting the antibody with the reducing agent at a concentration of between about 1 mM and about 40 mM.

27. The method of claim 20, wherein forming the thiolated specific-binding moiety comprises introducing a thiol group to the specific-binding moiety.

28. The method of claim 27, wherein introducing the thiol group to the specific-binding moiety comprises reacting the specific-binding moiety with a reagent selected from the group consisting of 2-Iminothiolane, SATA, SATP, SPDP, N-Acetylhomocysteinethiolactone, SAMSA, and cystamine, and combinations thereof.

29. The method of claim 27, wherein introducing the thiol group to the specific-binding moiety comprises reacting the specific-binding moiety with an oxidant to convert a sugar moiety of the specific-binding moiety into an aldehyde group and reacting the aldehyde group with cystamine.

30. The method of claim 29, wherein the oxidant comprises periodate ion, I₂, Br₂, and combinations thereof; or neuramimidase/galactose oxidase.

31. The method of claim 20, wherein reacting an amine group of a nanoparticle with a maleimide/active ester bifunctional PEG linker to form an activated nanoparticle comprises reacting the nanoparticle with a PEG maleimide/active ester having the formula: 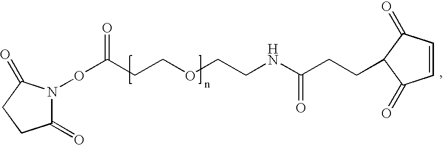
wherein n=1 to 50.

32. The method of claim 31, wherein n=4 to 12.

33. The method of claim 20, wherein the nanoparticle comprises a quantum dot.

34. A method for preparing an antibody-nanoparticle conjugate composition, comprising:
reacting an antibody with an oxidant to form an aldehyde-bearing antibody;
reacting the aldehyde-bearing antibody with a PEG maleimide/hydrazide bifunctional linker to form a thiol-reactive antibody; and
reacting the thiol-reactive antibody with a thiolated nanoparticle to form the antibody-nanoparticle conjugate.

35. The method of claim 34, wherein reacting an antibody with an oxidant to form the aldehyde-bearing antibody comprises oxidizing a glycosylated region of the antibody to form the aldehyde-bearing antibody.

36. The method of claim 35, wherein oxidizing a glycosylated region of the antibody comprises treating the antibody with periodate, I₂, Br₂, or a combination thereof, or neuramimidase/galactose oxidase.

37. The method of claim 35, further comprising forming the thiolated nanoparticle from a nanoparticle.

38. The method of claim 37, wherein forming the thiolated nanoparticle comprises introducing a thiol group to the nanoparticle.

39. The method of claim 38, wherein introducing the thiol group to the nanoparticle comprises reacting the nanoparticle with a reagent selected from the group consisting of 2-Iminothiolane, SATA, SATP, SPDP, N-Acetylhomocysteinethiolactone, SAMSA, and cystamine, and combinations thereof.

40. The method of claim 35, wherein reacting the aldehyde-bearing antibody with the PEG maleimide/hydrazide bifunctional linker to form the thiol-reactive antibody comprises reacting the aldehyde-bearing antibody with a linker having the formula: 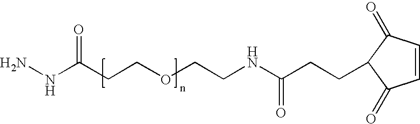
wherein n=1 to 50.

41. The method of claim 35, wherein the thiolated nanoparticle comprises a quantum dot.

42. The method of claim 35, wherein reacting an antibody with an oxidant to form an aldehyde-bearing antibody comprises introducing an average of between about 1 and about 10 aldehyde groups per antibody.

43. A method for detecting a molecule of interest in a biological sample, comprising:
contacting the biological sample with a specific-binding moiety-nanoparticle conjugate composition comprising a specific-binding moiety covalently coupled to a nanoparticle through a heterobifunctional PEG linker; and
detecting a signal generated by the conjugate bound to the molecule of interest.

44. The method of claim 43, wherein the biological sample comprises a tissue section or a cytology sample.

45. The method of claim 43, wherein the specific-binding moiety comprises an antibody or an avidin and the nanoparticle comprises a quantum dot.

46. The method of claim 43, wherein the specific-binding moiety comprises an antibody.

47. The method of claim 46, wherein the antibody is an anti-hapten antibody and the molecule of interest is a nucleic acid sequence detectable with a hapten-labeled probe sequence.

48. The method of claim 46, wherein the antibody comprises an anti-antibody antibody.

49. The method of claim 43, wherein the nanoparticle comprises a quantum dot and detecting comprises illuminating the biological sample with light of a wavelength that stimulates fluorescence emission by the quantum dot.

50. The method of claim 43, wherein at least two conjugates having different specific-binding moieties and separately detectable nanoparticles are contacted with the sample.

51. The method of claim 50, wherein the separately detectable nanoparticles comprise quantum dots having different emission wavelengths.
